(19) United States
(12) Patent Application Publication
(10) Pub. No.: US 2019/0209180 A1
(43) Pub. Date: Jul. 11, 2019

(54) THIN-FILM MICROMESH OCCLUSION DEVICES AND RELATED METHODS

(71) Applicant: Monarch Biosciences, Inc., Los Angeles, CA (US)

(72) Inventors: Colin Kealey, Los Angeles, CA (US); Vikas Gupta, Los Angeles, CA (US)

(21) Appl. No.: 16/357,112

(22) Filed: Mar. 18, 2019

Related U.S. Application Data
(63) Continuation of application No. PCT/US2017/051911, filed on Sep. 15, 2017.
(60) Provisional application No. 62/396,006, filed on Sep. 16, 2016.

(51) Int. Cl. A61B 17/12

(52) U.S. Cl. CPC A61B 17/12122; A61B 2017/00606; A61B 17/12168; A61B 17/12031

(57) ABSTRACT

A septal occlusion device for closing an abnormal opening in the heart includes a wire mesh support structure with a first disk, a second disk, and a waist portion joining the first and second disk; and a thin-film micromesh coupled to the wire mesh and configured to extend across the abnormal opening. A left arterial appendage (LAA) occlusion device for sealing an LAA in the heart includes a support structure having a plurality of struts extending radially from a center to a distal portion to form a substantially hemisphere or dome shape, the distal portion of each strut being configured to engage an interior wall of the left arterial appendage, and a thin-film micromesh cover attached to the support structure and configured to extend across the opening of the left arterial appendage.

### CROSS-REFERENCE TO RELATED APPLICATIONS

The present application is a continuation application of, and claims the benefit of, International Application No. PCT/US2017/051911, filed on Sep. 15, 2017, which claims the benefit of U.S. Provisional Application No. 62/396,006, filed on Sep. 16, 2016, which are both hereby incorporated by reference in their entirety.

### TECHNICAL FIELD

The present disclosure generally relates to thin-film micromesh medical devices and, more particularly, to thin-film micromesh occlusion devices for implantation in the heart.

### BACKGROUND

A septal occlusion device is a medical device used to close an abnormal opening in the wall of the heart (e.g., ventricular septal defects, atrial septal defects, patent ductus arteriosus, patent foramen ovale, or other openings in the wall of the heart). FIG. 1A is a schematic cross-sectional view of a septal occlusion device 100 and FIG. 1B is a top plan view of septal occlusion device 100. Septal occlusion device 100 includes a wire mesh structure 105 (e.g., a self-expandable braided wire mesh) forming an atrial disk 110, an atrial disk 115, and a waist portion 120 connecting atrial disk 110 and atrial disk 115. Septal occlusion device 100 may include one or more membranes 125 (e.g., a polyester membrane, a PTFE membrane, a PET membrane, or other polymer membrane) provided in atrial disk 110 and atrial disk 115, and a screw attachment 130 for attachment to a delivery cable. As shown in FIG. 1C, when implanted, septal occlusion device 100 may facilitate occlusion of an abnormal opening 135 at a wall of the heart 140. Membrane 125 may close abnormal opening 135 so that blood does not flow through abnormal opening 135 and may provide a substrate for tissue in-growth.

A left arterial appendage (LAA) occlusion device is a medical device used to seal off the left arterial appendage. As shown in FIG. 2A, an LAA occlusion device 200 may include a metal alloy frame 205 and a porous membrane covering 210 (e.g., a polyester membrane, a PTFE membrane, a PET membrane, or other polymer membrane) over a part of frame 205. As shown in FIG. 2B, when implanted, LAA occlusion device 200 is implanted at an LAA 220 of the heart. Membrane covering 210 may seal the LAA and provide a substrate for tissue growth to close off the LAA from the rest of the heart, which prevents blood clots generated at the LAA that may break loose and cause a stroke.

However, tissue growth on membrane 125 of septal occlusion device 100 or membrane covering 210 of LAA occlusion device 200 may take a long time (e.g. 45 days). Further, tissue growth on membrane 125 or membrane covering 210 may not provide a smooth tissue lining.

An additional advantage of a thin film based septal occlusion device over current devices is the ability to perform a septostomy subsequent to device placement. In certain limited circumstances, for example, in adults with pulmonary arterial hypertension and in pediatric patients with dextro-transposition of the great arteries, it is desirable to form a small hole between the left and right atria using minimally-invasive techniques. Prior treatment of a septal defect with current septal occlusion devices would preclude such a procedure because of the impermeable polymer-based membranes. A sufficiently porous thin film based septal occlusion device, however, would allow for a septostomy procedure post-implantation.

Thus, there is a need for improved occlusion devices for treatment of heart defects and sealing of the LAA.

### BRIEF DESCRIPTION OF THE DRAWINGS

FIG. 1A is a diagrammatic side view of a septal occlusion device.

FIG. 1B is a diagrammatic top plan view of the septal occlusion device of FIG. 1A.

FIG. 1C is a diagrammatic cross-sectional view of an abnormal opening in the wall of the heart in which the septal occlusion device of FIG. 1A is implanted to occlude the abnormal opening.

FIG. 2A is a diagrammatic side view of a left arterial appendage (LAA) occlusion device.

FIG. 2B is a diagrammatic cross-sectional view of an LAA of the heart in which the LAA occlusion device of FIG. 2A is implanted to seal the LAA.

FIG. 3A is a diagrammatic side view of a thin-film micromesh septal occlusion device with a thin-film micromesh provided in a braided wire structure according to an embodiment of the present disclosure.

FIG. 3B is a diagrammatic side view of a thin-film micromesh septal occlusion device with a thin-film micromesh cover according to an embodiment of the present disclosure.

FIG. 4 is a diagrammatic side view of a thin-film micromesh LAA occlusion device according to an embodiment of the present disclosure.

FIG. 5A is a diagrammatic plan view of a part of an etched semiconductor wafer for making a thin-film micromesh cover for an occlusion device.

FIG. 5B is a diagrammatic cross-sectional view of the wafer of FIG. 5A along lines D:D.

FIG. 6A is a diagrammatic perspective view of a portion of a thin-film micromesh cover prior to expansion.

FIG. 6B is a diagrammatic plan view of a portion of a thin-film micromesh cover after expansion.

FIG. 7 illustrates a method for forming the thin-film micromesh device of FIGS. 3A, 3B, or 4 using a three-dimensional thin-film micromesh according to an embodiment of the present disclosure.

FIG. 8 illustrates a method for forming the thin-film micromesh device of FIGS. 3A, 3B, or 4 using a two-dimensional thin-film micromesh according to an embodiment of the present disclosure.

FIG. 9A is an image showing results of a conventional braided stent implanted at a model aneurysm in a rabbit.

FIG. 9B is an image showing results of a thin-film Nitinol covered stent with a lower pore density implanted at a model aneurysm in a rabbit.

FIG. 9C is an image showing results of a thin-film Nitinol covered stent with a higher pore density implanted at the model aneurysm in a rabbit.

Embodiments of the present disclosure and their advantages are best understood by referring to the detailed description that follows. It should be appreciated that like reference numerals are used to identify like elements illustrated in one or more of the figures, in which the showings therein are for purposes of illustrating the embodiments and not for purposes of limiting them.

### DETAILED DESCRIPTION

One or more embodiments of the present disclosure provide improved occlusion devices that incorporate a fenestrated thin-film mesh and related methods. The thin-film mesh facilitates incorporation of the occlusion device into the surrounding tissue (e.g., heart tissue or endothelial tissue). More rapid incorporation of the occlusion device into the surrounding tissue may reduce healing time, and improved tissue incorporation may improve the seal formed by the device.

As used herein, a thin-film mesh (also referred to as a thin-film micromesh, a fenestrated thin-film micromesh, or a fenestrated thin-film micromesh sheet) is defined to be less than 100 microns in thickness (e.g., between 2 and 30 microns in thickness). An example thin-film micromesh comprises fenestrated thin-film Nitinol (TFN), although other thin-film micromesh materials may be used to form the occlusion device disclosed herein. The following discussion is thus directed to occlusion devices including thin-film Nitinol without loss of generality. Example fenestrated thin-film Nitinol is disclosed in International Application No. PCT/US2014/61836, filed on Oct. 22, 2014, which claims the benefit of U.S. Provisional Application No. 61/894,826, filed on Oct. 23, 2013 and U.S. Provisional Application No. 61/896,541, filed on Oct. 28, 2013; International Application No. PCT/US2016/039436, filed on Jun. 24, 2016, which claims the benefit of U.S. Provisional Application No. 62/185,513, filed on Jun. 26, 2015, U.S. Provisional Application No. 62/188,218, filed on Jul. 2, 2015, U.S. Provisional Application No. 62/209,185, filed on Aug. 24, 2015, U.S. Provisional Application No. 62/209,254, filed on Aug. 24, 2015, and U.S. Provisional Application No. 62/216,965, filed on Sep. 10, 2015; and International Application No. PCT/US2016/040864, filed on Jul. 1, 2016, which claims the benefit of U.S. Provisional Application No. 62/188,218, filed on Jul. 2, 2015, U.S. Provisional Application No. 62/209,185, filed on Aug. 24, 2015, U.S. Provisional Application No. 62/209,254, filed on Aug. 24, 2015, and U.S. Provisional Application No. 62/216,965, filed on Sep. 10, 2015. The contents of each of these applications are hereby incorporated by reference in their entirety.

To form a thin-film micromesh, Nitinol (NiTi) may be sputtered onto patterned silicon wafers. The patterned mesh may then be removed using a lift-off process by etching away a sacrificial layer such as a chromium layer to form a two-dimensional (2D) thin-film micromesh. A sheet of fenestrated thin-film Nitinol may be disposed about an occlusion device and attached, for example, by soldering, by an adhesive (e.g., glue), by fastening with a wire or string, and/or by stitches. Alternatively, this lift-off process is combined with multiple-layer depositions of Nitinol separated by layers of sacrificial material to fabricate, for example, a hemisphere shaped or cylindrical shaped thin-film micromesh, which are three-dimensional (3D) in the sense that two layers are joined together along their longitudinal edges such that the resulting joined layers may be opened up to form a cylinder.

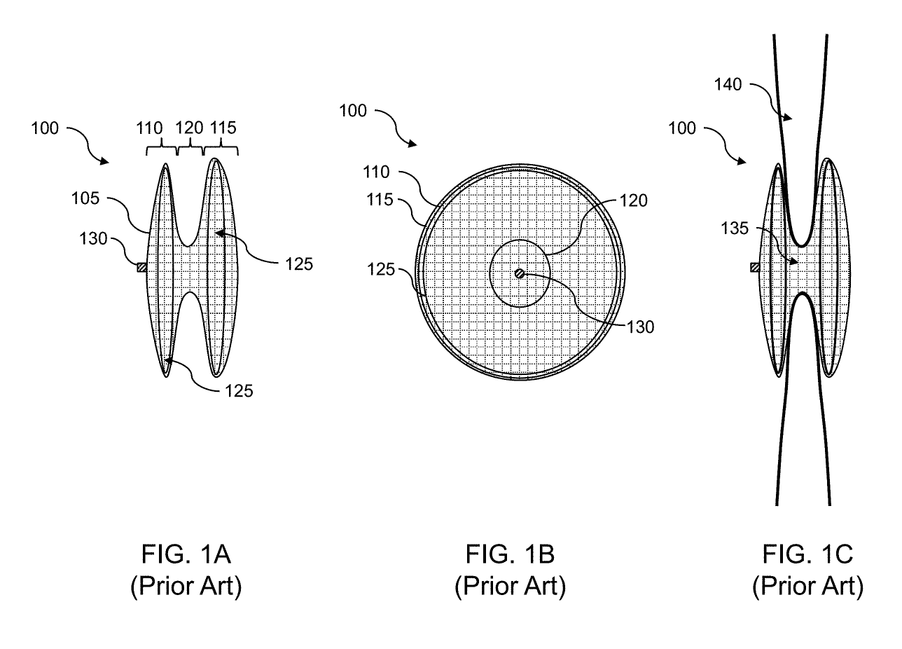

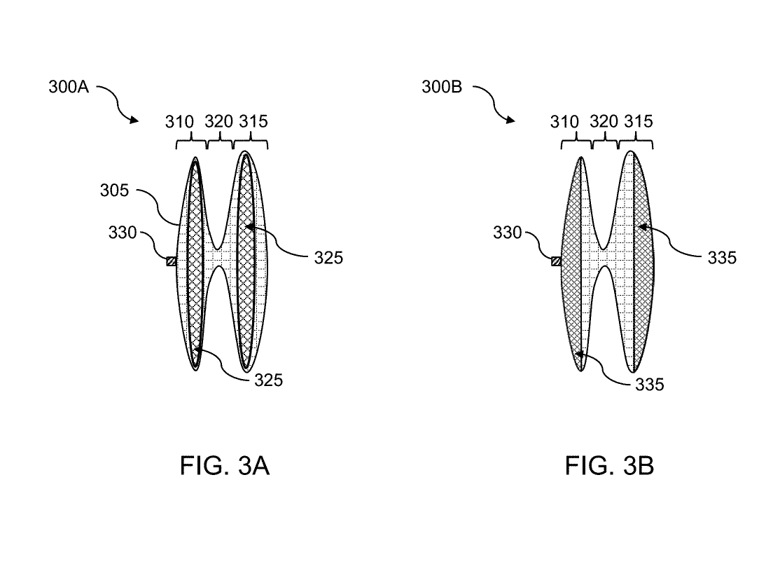

FIG. 3A is a diagrammatic side view of a thin-film micromesh septal occlusion device 300A with a thin-film micromesh 325 provided in a braided wire structure 305. Thin-film micromesh septal occlusion device 300A includes wire mesh structure 305 (e.g., a braided wire mesh) forming an atrial disk 310, an atrial disk 315, and a waist portion 320 connecting atrial disk 310 and atrial disk 315. Wire mesh structure 305 may be composed of a metal alloy (e.g., Nitinol alloy, a cobalt chromium, or other alloy). Thin-film micromesh septal occlusion device 300A may also include a screw attachment 330 for attachment to a delivery cable. Thin-film micromesh septal occlusion device 300A includes one or more thin-film micromeshes 325 disposed in atrial disk 310 and atrial disk 315 in place of polymer membrane 125 of conventional septal occlusion device 100 of FIGS. 1A-1C. Alternatively, thin-film micromesh septal occlusion device 300A includes one or more thin-film micromeshes 325 disposed in atrial disk 310 and atrial disk 315 in addition to polymer membrane 125 of conventional septal occlusion device 100 of FIGS. 1A-1C.

Thin-film micromesh 325 may be disposed inside wire mesh structure 305 without attachment to wire mesh structure 305. Alternatively, thin-film micromesh 325 is attached to a part of the inner surface of wire mesh structure 305. In one example, thin-film micromesh 325 is attached to wire mesh structure 305 by soldering (e.g., soldering with a low temperature solder), by fastening with a wire or string, by an adhesive (e.g., glue), or by stitches. In other examples, thin-film micromesh 325 is attached to wire mesh structure 305 using other fastening methods as appropriate.

FIG. 3B is a diagrammatic side view of a thin-film micromesh septal occlusion device 300A with thin-film micromesh covers 335. Similar to thin-film micromesh septal occlusion device 300A of FIG. 3A, thin-film micromesh septal occlusion device 300B includes a wire mesh structure 305 (e.g., a braided wire mesh) forming an atrial disk 310, an atrial disk 315, and a waist portion 320 connecting atrial disk 310 and atrial disk 315. Wire mesh structure 305 may be composed of a metal alloy (e.g., Nitinol alloy, cobalt chromium alloy, or other alloy). Thin-film micromesh septal occlusion device 300B may also include a screw attachment 330 for attachment to a delivery cable. Thin-film micromesh septal occlusion device 300B includes one or more thin-film micromesh covers 335 attached to atrial disk 310 and atrial disk 315, for example, at each end as shown in FIG. 3B. Thin-film micromesh covers 335 are attached to wire mesh structure 305 in place of thin-film micromesh 325 and/or polymer membrane 125 of conventional septal occlusion device 100 of FIGS. 1A-1C provided in wire mesh structure 305. Alternatively, thin-film micromesh covers 335 are attached to wire mesh structure 305 in addition to thin-film micromesh 325 and/or polymer membrane 125 of conventional septal occlusion device 100 of FIGS. 1A-1C provided in wire mesh structure 305.

Thin-film micromesh cover 335 may be attached to the outer surface of wire mesh structure 305. Alternatively, or in addition, thin-film micromesh cover 335 may be attached to the inner surface of wire mesh structure 305. In one example, thin-film micromesh cover 335 is attached to wire mesh structure 305 by soldering (e.g., soldering with a low temperature solder), by fastening with a wire or string, by an adhesive (e.g., glue), or by stitches. In other examples, thin-film micromesh cover 335 is attached to wire mesh structure 305 using other fastening methods as appropriate.

In other examples, mesh structure 305 of thin-film micromesh septal occlusion device 300A or 300B of FIGS. 3A-3B may be composed of a bioabsorbable metal or polymeric material that is absorbed, degraded, dissolved, or otherwise fully broken down after a predetermined amount of time (e.g., 3-6 months, 6-24 months, etc.) after implantation in a patient while thin-film micromesh 325 or thin-film micromesh cover 335 remains in the patient. By the time mesh structure 305 degrades, the abnormal opening may have fully healed and no longer require the mechanical support provided by mesh structure 305.

Thin-film micromesh septal occlusion devices 300A and 300B are shown in their deployed state in FIGS. 3A and 3B. Thin-film micromesh septal occlusion device 300A, 300B may be crimped to a retracted state and placed in a delivery device. Delivery device may be used to place thin-film micromesh septal occlusion device 300A, 300B at an opening at the heart, and thin-film micromesh septal occlusion device 300A, 300B may be deployed such that waist portion 320 is placed at or engages the opening and atrial disk 310 is on one side of the opening and atrial disk 315 is on the opposing side of the opening.

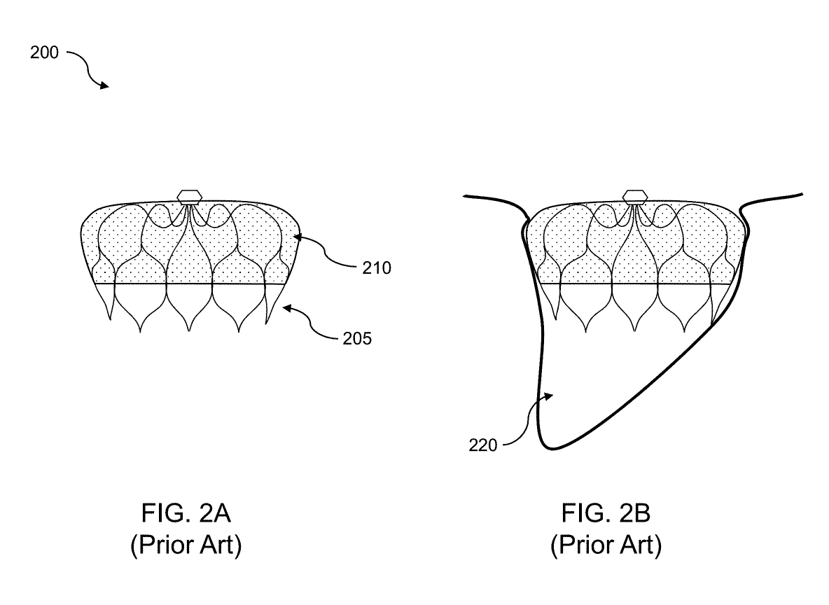

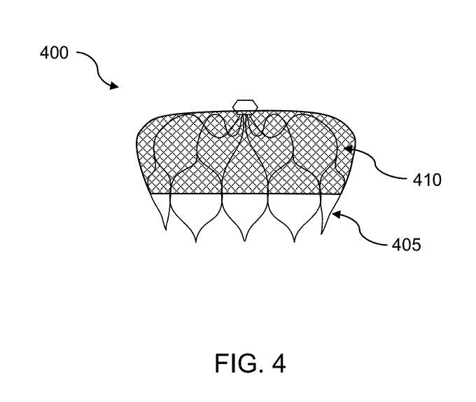

FIG. 4 is a diagrammatic side view of a thin-film micromesh left arterial appendage (LAA) occlusion device 400. Thin-film micromesh LAA occlusion device 400 includes a support structure or frame 405 (e.g., a metal alloy frame consisting of Nitinol alloy, cobalt chromium alloy, or other alloy) and a Nitinol micromesh cover 410 attached to frame 405. Nitinol micromesh cover 410 is attached over a part of frame 405 in place of polymer membrane covering 210 of conventional LAA occlusion device 200 of FIGS. 2A-2B. Alternatively, Nitinol micromesh cover 410 is attached over a part of frame 401 in addition to porous membrane covering 210 (e.g., a polyester membrane, a PTFE membrane, a PET membrane, or other polymer membrane) of conventional LAA occlusion device 200 of FIGS. 2A-2B.

In other examples, frame 405 of LAA occlusion device 400 of FIG. 4 may be composed of a bioabsorbable metal or polymeric material that is absorbed, degraded, dissolved, or otherwise fully broken down after a predetermined amount of time (e.g., 3-6 months, 6-24 months, etc.) after implantation in a patient while thin-film micromesh cover 410 remains in the patient. By the time frame 405 degrades, the LAA may have fully sealed and no longer require the mechanical support provided by frame 405.

LAA occlusion device 400 is shown in its deployed state in FIG. 4. LAA occlusion device 400 may be crimped to a retracted state and placed in a delivery device. Delivery device may be used to place LAA occlusion device 400 to the LAA and LAA occlusion device may be deployed such that the radially extending struts of LAA occlusion device 400 engages the interior wall of the LAA.

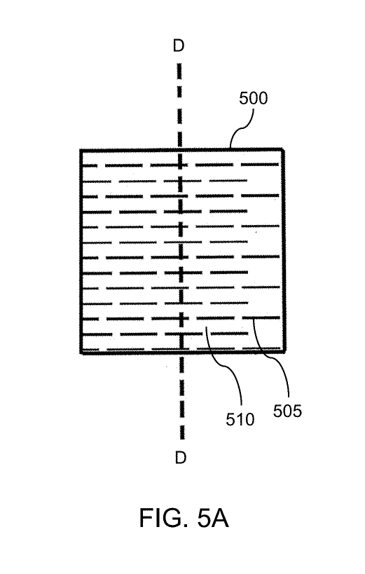

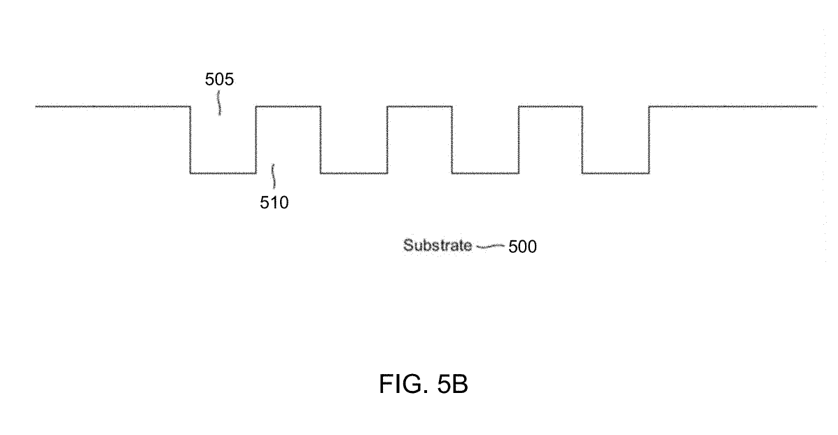

In one embodiment, a thin-film micromesh such as thin-film micromesh 325, thin-film micromesh cover 335, or thin-film micromesh cover 410 may be formed using a deep-reactive ion etched semiconductor wafer as described International Application Nos. PCT/US2014/61836, PCT/US2016/039436, and International Application No. PCT/US2016/040864, previously referenced herein. FIG. 5A is a diagrammatic plan view of a part of a substrate such as an etched wafer 500 formed by a deep reactive-ion etching (DRIE) process. Grooves 505 are separated by lands 510. Rows of grooves 505 are displaced with respect to adjacent rows of grooves 505 such that a groove 505 in one row is longitudinally displaced by approximately 50% with regard to the neighboring grooves in the immediately-adjacent grooves. FIG. 5B is a diagrammatic cross-section view of etched wafer 500 of FIG. 5A along line D:D. Grooves 505 are separated by lands 510. The width of lands 510 may be 1 to 30 microns (e.g., between 4 and 30 microns, between 4 and 20 microns, between 1 and 20 microns, approximately 10 microns, etc.). Similarly, the width of grooves 505 may be 1 to 30 microns (e.g., between 4 and 30 microns, between 4 and 20 microns, between 1 and 20 microns, approximately 10 microns, etc.). The longitudinal extent of each groove 505 may range from a few microns to approximately 500 microns (e.g., between 100 microns and 500 microns, between 100 microns and 400 microns, between 100 microns and 300 microns, between 150 microns and 400 microns, etc.).

Nitinol may then be deposited on etched wafer 500 to a thickness of approximately 1 to 30 microns (e.g., between 4 and 30 microns, between 4 and 20 microns, between 2 and 20 microns, approximately 10 microns, etc.) and then lifted off. Grooves 505 will then be duplicated on the resulting patterned thin-film Nitinol sheet as corresponding longitudinally-extending fenestrations. The resulting patterns of fenestrations may also be denoted as a fiche in that the fenestrations are in collapsed form prior to an expansion of the Nitinol sheet. Just like a microfiche, each fiche or pattern of fenestrations effectively codes for the resulting fenestrations when the stent cover is expanded to fully open up the fenestrations.

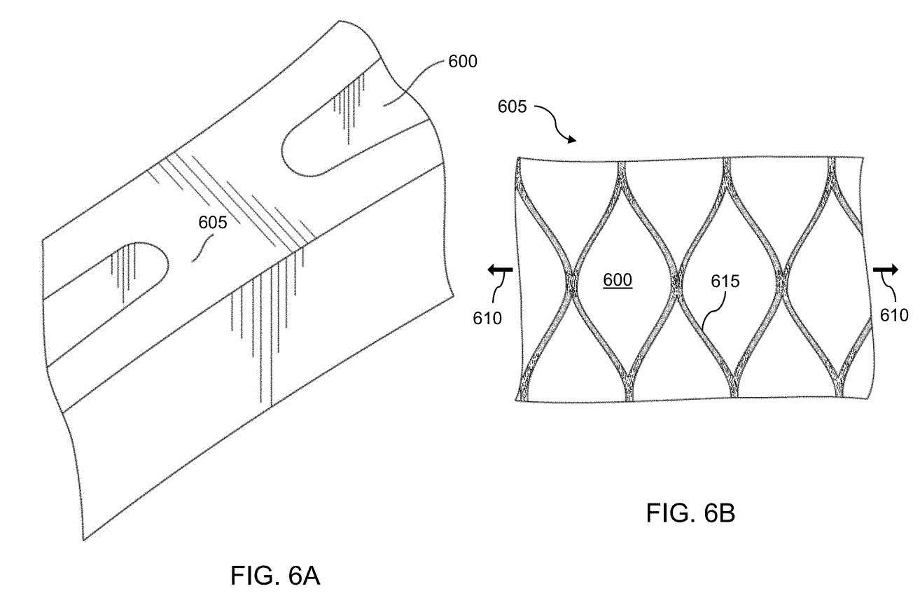

This may be better appreciated with regard to FIG. 6A, which shows two fenestrations 600 in a portion of a thin-film micromesh 605 (e.g., thin-film micromesh 325, thin-film micromesh cover 335, or thin-film micromesh cover 410) prior to expansion. In FIG. 6B, mesh 605 is expanded in the lateral direction 610 (also referred to as the axis of expansion of mesh 605) orthogonal to the longitudinal axis of fenestrations 600 (also referred to as the longitudinal direction or long axis of fenestrations 600) such that fenestrations 600 open up into a “chain-link” fence pattern of diamond-shaped fenestrations. It will be appreciated that other fenestration shapes may be used in alternative embodiments. In some embodiments, the expansion may extend mesh 605 in a range from 50% to 800%. Thin-film micromesh 605 as fabricated (prior to expansion) has fenestrations 600 that duplicate grooves 505 of wafer 500, and struts 615 that duplicate lands 510 of wafer 500. Accordingly, prior to expansion, the longitudinal extent of each fenestration 600 may range from a few microns to approximately 500 microns (e.g., between 100 microns and 500 microns, between 100 microns and 400 microns, between 100 microns and 300 microns, between 150 microns and 400 microns, etc.). After expansion, the longitudinal extent of each fenestration 600 decreases (e.g., between 5% and 20%) while the width of each fenestration 600 increases (e.g., between 100 to 800%). Struts 615 may have a thickness of between 1 and 30 microns (e.g., between 4 and 30 microns, between 4 and 20 microns, between 2 and 20 microns, approximately 10 microns, etc.) prior to and after expansion.

The resulting high pore density, fenestrations per square mm, (e.g., between 81 and 1075 pores per mm², between 134 and 227 pores per mm², between 81 and 227 pores per mm², etc.) and low metal coverage (e.g., between 19 and 66%, between 24 and 36%, between 19% and 36%, etc.) is very advantageous with regard to promoting a planar deposition of fibrin and a rapid tissue in-growth. In this fashion, the thin-film micromesh is incorporated into the surrounding tissue (e.g., heart tissue or endothelial tissue), which thus seals the abnormal opening or the LAA.

Thin-film micromeshes such as thin-film micromesh 605, orientation of fenestrations, and various parameters for thin-film micromeshes relating to fenestrations such as fenestrations 605, struts such as struts 615, pore density, percent metal coverage, strut angle, and other features of the thin-film micromeshes may be implemented in accordance with the techniques described in International Application Nos. PCT/US2014/61836, PCT/US2016/039436, and International Application No. PCT/US2016/040864, previously referenced herein.

In addition to sealing the abnormal opening or the LAA, the biological seal of the tissue ingrowth also serves to anchor the thin-film micromesh occlusion device (e.g., device 300A, 300B, or 400). As the body incorporates the thin-film Nitinol elements of thin-film occlusion device 200 into the vessel wall, the thin-film occlusion device is stabilized mechanically, thereby mitigating the issue of migration. Notably, this is accomplished without damage to the vessel wall or adjacent structures.

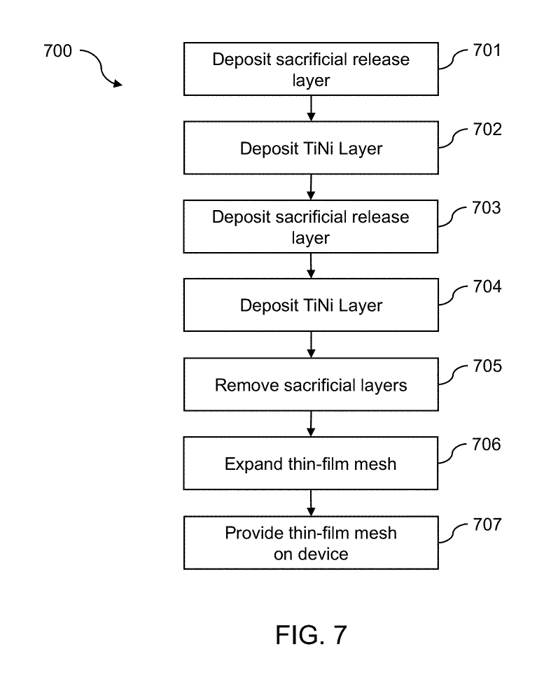

FIG. 7 illustrates a method 700 for forming a thin-film occlusion device such as device 300A, 300B, or 400 using a three-dimensional thin-film micromesh.

At block 701, a first sacrificial layer (e.g., a lift-off or release layer) of Cr (or other sacrificial or barrier layers) is deposited on a silicon substrate (e.g., silicon wafer substrate 500), for example, in a sputtering chamber while the substrate is held at high vacuum or under ultra-high vacuum, using e-beam evaporation or PECVD. When subsequently etched away, the lift-off layer may release the finished product such as thin-film micromesh 325, 335, or 410 from the substrate (e.g., silicon wafer substrate 500) and may thus be referred to as a release layer. The lift-off layer may be 1700 to 3000 Angstroms of sputter-deposited chromium. Block 701 and one or more of subsequent blocks 702 through 704 may all be performed while the substrate continues to be held under a vacuum in a sputtering chamber and without removing the vacuum (or removing the substrate wafer or device from the vacuum chamber) until all depositions are completed.

Prior to the deposition of the lift-off layer, the substrate may first (e.g., before deposition) be prepared in block 701 by etching (using, for example, dry etching or DRIE) grooves or trenches that will correspond to fenestrations 600 of the web fiche pattern or other surface features that may correspond to structures (e.g., mesh fenestrations) of the finished product.

At block 702, a first layer of NiTi may be deposited using one or more sputtering or other techniques. An example thickness of this first layer (as well as the second layer of NiTi) is between 2 and 30 microns in thickness (e.g., 3 to 5 microns).

At block 703, a second sacrificial layer of Cr (or other sacrificial or barrier layers) may be deposited on the silicon substrate (e.g., silicon wafer substrate 500), for example, in a sputtering (or vacuum) chamber while the substrate continues to be held at high vacuum or under ultra-high vacuum, using e-beam evaporation or PECVD. A shadow mask may be placed over the substrate and the previously deposited layers such as the release layer and the first NiTi layer prior to depositing the second sacrificial layer to protect covered (or blocked) areas from deposition of the second Cr sacrificial layer (or other sacrificial or barrier layers). The shadow mask may be removed from the substrate and the accumulated deposited layers after depositing the second sacrificial layer.

In some embodiments, an aluminum bonding layer is applied using a reverse mask to prevent formation of an oxidized surface layer on the first NiTi layer. It will be appreciated that bonding of one NiTi layer onto another can be problematic if an oxidized surface layer is formed on the first NiTi layer because this surface layer inhibits the bonding of one NiTi layer to another. The reverse mask (as implied by the name) is the complement of the shadow mask used to form the second sacrificial layer. In other words, the reverse mask covers the second sacrificial layer and exposes the uncovered areas of the first NiTi layer. Aluminum may then be sputtered through the reverse mask to form the bonding layer. Since the bonding layer is applied, the first NiTi layer may be exposed to the atmosphere between the masking with the shadow mask and the subsequent masking with the reverse mask. In this fashion, manufacturing costs are lowered in that the applications of the masks is greatly aided by performing the mask applications outside of the vacuum chamber using, for example, conventional semiconductor pick-and-place equipment. Alternatively, the first NiTi layer may be maintained in a vacuum or an ultra-high vacuum until a second layer of NiTi is deposited, including during the application and removal of the shadow mask.

At block 704, a second layer of NiTi may be deposited using one or more sputtering or other techniques. At this block, deposition of the second layer of NiTi may result in the second layer of NiTi bonding to the first layer of NiTi at those areas left exposed by the second sacrificial layer, forming, for example, bonds at the edges of the thin-film micromesh.

In embodiments in which the bonding layer is utilized, wafer 500 may be heated to approximately 500 to 600 degrees prior to removal of the lift-off and sacrificial layers at block 706. Such heating partially melts the aluminum, which then becomes very reactive despite the formation of some aluminum oxides. The molten un-oxidized aluminum is very reactive and chemically bonds to the NiTi layers, resulting in a very secure bond, despite the formation of an oxidized NiTi surface on the first NiTi layer.

At block 705, removal of the sacrificial layers (e.g., the first sacrificial or release layer and the second sacrificial layer) may be performed using a wet etch and may be performed after allowing the vacuum chamber to repressurize or after removing substrate 500 from the vacuum chamber. Etching the sacrificial layers may release the thin-film micromesh from the substrate and may remove interior layers such as the second sacrificial layer. The etch may comprise soaking silicon substrate wafer 500 and the deposited layers in a solution, for example, of Cr etch, and may create a lumen where sacrificial layers are removed between the first and second NiTi layers that are joined at the edges.

At block 706, the thin-film micromesh is expanded such that fenestrations 600 open up into a “chain-link” fence pattern of diamond-shaped fenestrations. Further processing may be performed, such as shaping the thin-film micromesh including, for example, shaping the thin-film micromesh into a more hemisphere shape or cylindrical shape using a mandrel. With the thin-film micromesh in the desired shape, the NiTi layers may be crystallized. Blocks 701-706 are further described in International Application Nos. PCT/US2014/61836, PCT/US2016/039436, and International Application No. PCT/US2016/040864, previously referenced herein.

At block 707, the thin-film micromesh (e.g., thin-film micromesh 325, 335, or 410) is attached or otherwise provided on an occlusion device to form a thin-film micromesh occlusion device (e.g., thin-film micromesh occlusion device 325, 335, or 410). The thin-film occlusion device may then be implanted in a patient using a delivery system.

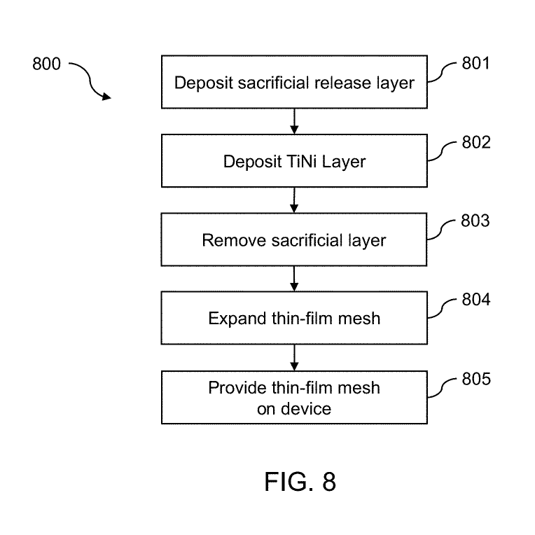

FIG. 8 illustrates a method 800 for forming a thin-film micromesh occlusion device such as device 300A, 300B, or 400 using two-dimensional thin-film micromeshes.

At block 801, a sacrificial layer (e.g., a lift-off or release layer) of Cr (or other sacrificial or barrier layers) is deposited on a silicon substrate (e.g., silicon wafer substrate 500), for example, in a sputtering chamber while the substrate is held at high vacuum or under ultra-high vacuum, using e-beam evaporation or PECVD. Prior to the deposition of the lift-off layer, the substrate may first (e.g., before deposition) be prepared in block 801 by etching (using, for example, dry etching or DRIE) grooves or trenches that will correspond to fenestrations 600 of the web fiche pattern or other surface features that may correspond to structures (e.g., mesh fenestrations) of a finished product such as thin-film micromesh 325, 335, or 410.

At block 802, a layer of NiTi may be deposited using one or more sputtering or other techniques. An example thickness of this first layer (as well as the second layer of NiTi) is between 2 and 30 microns in thickness (e.g., 3 to 5 microns).

At block 803, removal of the sacrificial layers may be performed using a wet etch and may be performed after allowing the vacuum chamber to repressurize or after removing substrate 500 from the vacuum chamber. Etching the sacrificial layers may release the thin-film micromesh from the substrate. The etch may comprise soaking silicon substrate wafer 500 and the deposited layers in a solution, for example, of Cr etch.

At block 804, the thin-film micromesh is expanded such that fenestrations 600 open up into a “chain-link” fence pattern of diamond-shaped fenestrations. Further processing may be performed, such as shaping the thin-film micromesh including, for example, shaping the thin-film micromesh into a more cylindrical shape by annealing on a mandrel. With the thin-film micromesh in the desired shape, the NiTi layers may be crystallized.

At block 805, the thin-film micromesh (e.g., thin-film micromesh 325, 335, or 410) is attached or otherwise provided on an occlusion device to form a thin-film micromesh occlusion device (e.g., thin-film, micromesh occlusion device 325, 335, or 410). The thin-film occlusion device may then be implanted in a patient using a delivery system.

The thin-film micromesh formed using the techniques described herein is planar with regard to the wire intersections. In that regard, the columnar fenestrations may be expanded into diamond shapes (e.g., having a length of approximately 300 microns and a width of approximately 150 microns). In contrast, the resulting wire forming the diamond-shaped fenestrations is only 2 to 30 microns in thickness. Each “corner” of the diamond-shaped fenestration is thus relatively flat, such that a null region with regard to fluid flow is formed at each corner. This may be better appreciated with regard to FIG. 6B, which shows the diamond-shaped fenestrations that result upon expansion. As shown in the close-up view in FIG. 6A, for the adjacent longitudinal ends of two diamond-shaped fenestrations 600, the thin-film micromesh 605 forms flat interstices that are advantageously conducive to the desired clotting process so that flow diversion of aneurysm is safely achieved. Such interstices are absent in a conventional wire mesh because of the weaving of the relatively coarse wire.

Occlusion devices that include thin-film Nitinol meshes facilitate robust endothelialization and tissue in-growth and, as such, thin-film Nitinol meshes may be advantageously used to improve occlusion devices. A conventional braided stent, a thin-film Nitinol covered stent with a lower pore density, and a thin-film Nitinol covered stent with a higher pore density were tested by implanting in model aneurysms created in rabbits. The animals were then sacrificed after several weeks, and the degree of aneurysm neck healing was examined by removing the arterial vessel segments containing the devices and the model aneurysms for pathological analysis. For the pathological analysis, the arterial vessels were cut along their long axes generating two approximately equal halves, with one half containing the model aneurysm. The sections with the model aneurysm were analyzed with light microscopy. The sections of the devices and micromesh covering the aneurysm neck region were the primary areas of interest.

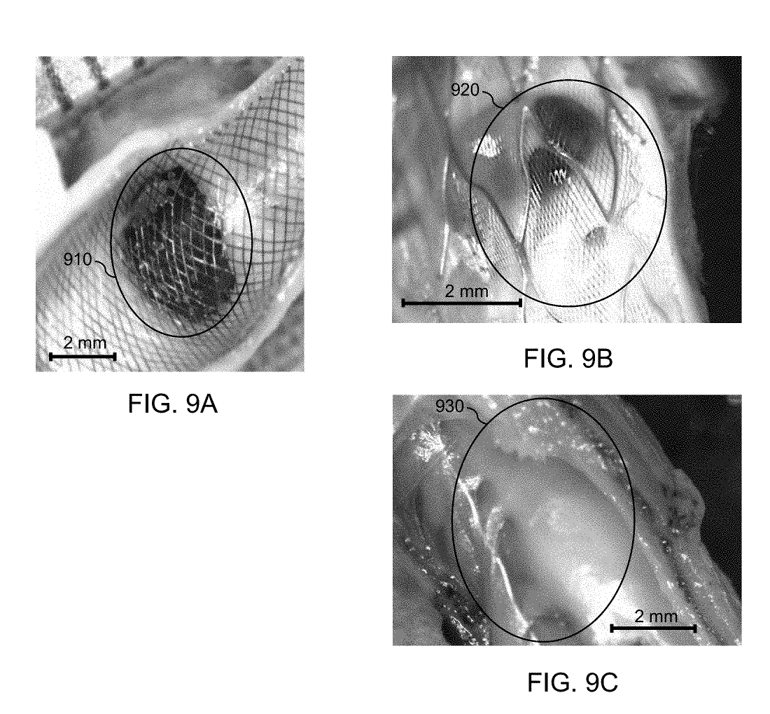

FIG. 9A is an image showing results of the conventional braided stent 4 weeks after implanting at the model aneurysm in a rabbit. The conventional braided stent had a pore density of about 14 pores/mm² as implanted.

FIG. 9B is an image showing results of the thin-film Nitinol covered stent having a lower pore density 8 weeks after implanting at the model aneurysm in a rabbit. The thin-film Nitinol was fabricated with a slit length of approximately 300 μm. The thin-film Nitinol had a pore density of approximately 70 pores/mm² as implanted. The thin-film Nitinol had a pore density may range from 38 to 70 pores/mm² when the strut angle (angle between two struts) is between 30 and 90 degrees. The thin-film Nitinol had a percent metal coverage of between 14% and 21%, and an edge density of between 23 mm of edge per mm² of surface area and 42 mm of edge per mm² of surface area.

FIG. 9C is an image showing results of the thin-film Nitinol covered stent having a higher pore density 8 weeks after implanting at the model aneurysm in a rabbit. The thin-film Nitinol of this device was fabricated with a slit length of approximately 150 μm. The thin-film Nitinol had a pore density of approximately 150 pores/mm² as implanted. The pore density of the thin-film Nitinol may range from 134 to 227 pores/mm² when the strut angle is between 30 and 90 degrees. The thin-film Nitinol had a percent metal coverage of between 24% and 36%, and an edge density of between 40 mm of edge per mm² of surface area and 68 mm of edge per mm² of surface area.

The aneurysm neck area 920 of the low-pore density thin-film Nitinol covered stent and the aneurysm neck area 930 of the high-pore density thin-film Nitinol covered stent both had robust endothelialization and tissue in-growth compared to the aneurysm neck area 910 of the conventional braided stent. Further, the aneurysm neck area 930 of the high-pore density thin-film Nitinol covered stent had improved endothelialization and tissue in-growth compared to the aneurysm neck area 920 of low-pore density thin-film Nitinol covered stent. Advantageously, thin-film micromesh cover 215 composed of thin-film Nitinol having a pore density of between 50 and 500 pores/mm² (e.g., between 50 and 250 pores/mm²) will facilitate rapid incorporation of a thin-film incorporated occlusion device such as thin-film occlusion device 200 into surrounding tissue.

Embodiments described herein illustrate but do not limit the disclosure. It should also be understood that numerous modifications and variations are possible in accordance with the principles of the present disclosure. Accordingly, the scope of the disclosure is best defined only by the following claims.

What is claimed is:

1. An occlusion device for closing an opening in a heart, comprising:
a support structure configured to engage the opening; and
at least one fenestrated thin-film micromesh coupled to the support structure and configured to extend across the opening of the heart.

2. The occlusion device of claim 1, wherein the support structure comprises a wire mesh comprising:
a first disk having a first portion extending radially from a central point at one end of the wire mesh to a first outer radius, and a second portion tapering from the first portion at the first outer radius to a first inner radius less than the first outer radius;
a second disk having a third portion extending radially from a central point at an opposite end of the wire mesh to a second outer radius, and a fourth portion tapering from the third portion at the second outer radius to a second inner radius less than the second outer radius; and
a waist portion joining the second portion at the first inner radius and the fourth portion at the second inner radius.

3. The occlusion device of claim 2, wherein the at least one fenestrated thin-film micromesh comprises a first fenestrated thin-film micromesh disposed in the first disk, and a second fenestrated thin-film micromesh disposed in the second disk.

4. The occlusion device of claim 2, wherein the at least one fenestrated thin-film micromesh comprises:
a first fenestrated thin-film micromesh cover attached to an outer surface of the first portion of the first disk, the first fenestrated thin-film micromesh cover having a shape corresponding to the outer surface of the first portion and covering the first portion; and
a second fenestrated thin-film micromesh cover attached to an outer surface of the third portion of the second disk, the second fenestrated thin-film micromesh having a shape corresponding to a surface of the third portion and covering the third portion.

5. The occlusion device of claim 1, wherein the at least one fenestrated thin-film micromesh comprises at least one fenestrated thin-film Nitinol micromesh, and wherein the support structure is a Nitinol alloy wire mesh.

6. The occlusion device of claim 1, wherein the at least one fenestrated thin-film micromesh comprises at least one two-dimensional fenestrated thin-film micromesh, at least one three-dimensional fenestrated thin-film micromesh, or both.

7. The occlusion device of claim 1, wherein the at least one fenestrated thin-film micromesh has a thickness of between 2 and 20 microns, wherein each fenestration of the at least one fenestrated thin-film micromesh has a length of between 25 and 500 microns along a long axis of the fenestration, wherein each strut of the at least one fenestrated thin-film micromesh has a width of between 4 microns and 30 microns, and the at least one fenestrated thin-film micromesh has a pore density of between 50 and 2000 pores/mm².

8. An occlusion device for sealing a left arterial appendage, comprising:
a support structure configured to engage an interior wall of the left arterial appendage; and
a fenestrated thin-film micromesh cover attached to the support structure and configured to extend across the opening of the left arterial appendage.

9. The occlusion device of claim 8, wherein the support structure comprises a plurality of struts extending radially from a center to a distal portion to form a substantially hemisphere or dome shape, wherein the distal portion of each strut is configured to engage the interior wall of the left arterial appendage.

10. The occlusion device of claim 8, wherein the fenestrated thin-film micromesh cover comprises a fenestrated thin-film Nitinol sheet, and wherein the support structure is a Nitinol alloy frame.

11. The occlusion device of claim 8, wherein the fenestrated thin-film micromesh cover comprises a two-dimensional fenestrated thin-film micromesh sheet.

12. The occlusion device of claim 8, wherein the fenestrated thin-film micromesh cover comprises a three-dimensional fenestrated thin-film micromesh cover having a substantially hemisphere or dome shape corresponding to a part of the substantially hemisphere or dome shape of the support structure.

13. The occlusion device of claim 8, wherein the fenestrated thin-film micromesh cover has a thickness of between 2 and 20 microns, wherein each fenestration of the fenestrated thin-film micromesh cover has a length of between 100 and 500 microns along a long axis of the fenestration, wherein each strut of the fenestrated thin-film micromesh cover has a width of between 4 microns and 30 microns, wherein the fenestrated thin-film micromesh cover has a density of between 50 and 500 pores/mm², and wherein the fenestrated thin-film micromesh cover has a density of between 50 and 500 pores/mm².

14. A method, comprising:
forming a fenestrated thin-film micromesh sheet; and
coupling the fenestrated thin-film micromesh sheet to a support structure configured to engage an opening or a cavity in the heart to form a thin-film micromesh occlusion device for implantation in the heart to occlude the opening or the cavity.

15. The method of claim 14, wherein the fenestrated thin-film micromesh sheet comprises Nitinol, and wherein the forming of the fenestrated thin-film micromesh sheet comprises:
deep reactive ion etching a pattern of grooves on a surface of a substrate, the grooves corresponding to fenestrations in a desired Nitinol structure;
depositing a lift-off layer on the grooved substrate surface;
depositing a first Nitinol layer over the lift-off layer;
lifting off the fenestrated thin-film micromesh sheet by etching, wherein the etching removes the lift-off layer; and
expanding the fenestrated thin-film micromesh sheet to expand the fenestrations.

16. The method of claim 15, wherein the forming of the fenestrated thin-film micromesh sheet further comprises:
depositing a sacrificial layer over the first Nitinol layer; and
depositing a second Nitinol layer over the sacrificial layer;
wherein the etching further removes the sacrificial layer, and wherein the forming of the fenestrated thin-film micromesh sheet comprises forming a three-dimensional fenestrated thin-film micromesh sheet.

17. The method of claim 15, wherein:
the deep reactive ion etching the pattern of the grooves comprises forming the grooves having a length of between 25 microns and 500 microns such that each fenestration of the thin-film micromesh sheet has a length of between 25 and 500 microns before the expanding, each row of grooves being spaced apart from an adjacent row of grooves by between 4 and 30 microns such that each strut of the thin-film micromesh sheet has a width of between 4 microns and 30 microns; and
the depositing comprises depositing the first Nitinol layer having a thickness of between 2 and 30 microns such that the fenestrated thin-film micromesh sheet has a thickness of between 2 and 30 microns.

18. The method of claim 14, wherein the attaching of the thin-film micromesh sheet comprises attaching the thin-film micromesh sheet to an outer surface of the support structure by low-temperature soldering, by using an adhesive, or by using wire or string.

19. The method of claim 14, wherein the expanding comprises expanding the fenestrated thin-film micromesh sheet such that the fenestrated thin-film micromesh sheet has a density of between 50 and 2000 pores/mm².

20. The method of claim 14, further comprising:
implanting the thin-film micromesh occlusion device at the heart to close an opening or seal a left arterial appendage.
